(19) United States
(12) Patent Application Publication
(10) Pub. No.: US 2008/0255420 A1
(43) Pub. Date: Oct. 16, 2008

(54) Surgical instrument

(75) Inventors: Woojin Lee, Hopkinton, MA (US); Andres Chamorro, Natick, MA (US)

Correspondence Address: David M. Driscoll, Esq., 1201 Canton Avenue, Milton, MA, 02186, US

(73) Assignee: Cambridge Endoscopic Devices, Inc.

(21) Appl. No.: 11/786,167

(22) Filed: Apr. 10, 2007

(51) Int. Cl. A61B 1/313

(52) U.S. Cl. 600/137

(57) ABSTRACT

A surgical instrument having an instrument shaft having proximal and distal ends, a tool disposed from the distal end of the instrument shaft, a control handle coupled from the proximal end of the instrument shaft, a distal motion member for coupling the distal end of the instrument shaft to the tool, a proximal motion member for coupling the proximal end of the instrument shaft to the handle and actuation means extending between distal and proximal motion members for coupling motion of the proximal motion member to the distal motion member for controlling the positioning of the tool. Rotation control and locking members are also disclosed.

### TECHNICAL FIELD

The present invention relates in general to medical instruments, and more particularly to manually-operated surgical instruments that are intended for use in minimally invasive surgery or other forms of surgical or medical procedures or techniques. The instrument described herein is primarily for a laparoscopic procedure, however, it is to be understood that the instrument of the present invention can be used for a wide variety of other procedures, including intraluminal procedures.

### BACKGROUND OF THE INVENTION

Endoscopic and laparoscopic instruments currently available in the market are extremely difficult to learn to operate and use, mainly due to a lack of dexterity in their use. For instance, when using a typical laparoscopic instrument during surgery, the orientation of the tool of the instrument is solely dictated by the location of the target and the incision. These instruments generally function with a fulcrum effect using the patients own incision area as the fulcrum. As a result, common tasks such as suturing, knotting and fine dissection have become challenging to master. Various laparoscopic instruments have been developed over the years to overcome this deficiency, usually by providing an extra articulation often controlled by a separately disposed control member for added control. However, even so these instruments still do not provide enough dexterity to allow the surgeon to perform common tasks such as suturing, particularly at any arbitrarily selected orientation. Also, existing instruments of this type do not provide an effective way to hold the instrument in a particular position. Moreover, existing instruments require the use of both hands in order to effectively control the instrument.

Accordingly, an object of the present invention is to provide an improved laparoscopic or endoscopic surgical instrument that allows the surgeon to manipulate the tool end of the surgical instrument with greater dexterity.

Another object of the present invention is to provide an improved surgical or medical instrument that has a wide variety of applications, through incisions, through natural body orifices or intraluminally.

A further object of the present invention is to provide an improved medical instrument that is characterized by the ability to lock the instrument in a pre-selected particular position.

Another object of the present invention is to provide a locking feature that is an important adjunct to the other controls of the instrument enabling the surgeon to lock the instrument once in the desired position. This makes it easier for the surgeon to thereafter perform surgical procedures without having to, at the same time, hold the instrument in a particular bent configuration.

Still another object of the present invention is to provide an improved medical instrument that can be effectively controlled with a single hand of the user.

A further object of the present invention is to provide a medical instrument in which the distal motion member is formed of a ball and socket structure having three dimensional movement.

Still another object of the present invention is to provide a medical instrument in which there is provided a means to readily convert the instrument tip into a substantially rigid tip.

### SUMMARY OF THE INVENTION

To accomplish the foregoing and other objects, features and advantages of the present invention there is provided, in one embodiment thereof, an instrument having a proximal control handle and a distal tool that are intercoupled by an elongated instrument shaft, proximal and distal movable members that respectively intercouple the proximal control handle and the distal tool with the instrument shaft, cabling that extends between the movable members so that a motion at the proximal movable member controls the distal movable member, and a control member at the control handle and manipulable by a user to control, via the proximal and distal movable members, the rotation of the distal tool about its distal tool axis. The control member comprises a slide member mounted on the handle.

In accordance with other aspects of the invention the slide member moves longitudinally to, in turn, control the rotation about a distal tool axis that is a longitudinal axis common to both the distal movable member and said tool; the proximal movable member may comprise a proximal bendable member; an angle locking means may be provided on the handle for locking the angle between the proximal and distal movable members; the movable members may be bendable members and the control member may further comprise gear means for translating linear sliding motion into rotation of the proximal bendable member and the movable members may be bendable members and the control member may further comprise a pulley and capstan means for translating linear sliding motion into rotation of the proximal bendable member.

In accordance with another embodiment the medical instrument has a proximal control handle and a distal tool that are intercoupled by an elongated instrument shaft that is meant to pass internally of an anatomic body, proximal and distal movable members respectively intercouple the proximal control handle and the distal tool with the instrument shaft, cable actuation means is disposed between the movable members, for controlling the positioning of the distal tool, and a locking mechanism is provided having locked and unlocked positions, disposed about the proximal movable member and manually controlled so as to fix the position of the proximal movable member relative to the handle in the locked position thereof, said locking mechanism comprising a socket and a split ball within the socket.

In accordance with still other aspects of the invention the locking mechanism may further comprise a wedge member that engages with the split ball; a slide member may be provided for actuating the wedge member which, in turn, expands the split ball to contact the socket; the proximal movable member may be a bendable member and the slide member may be mounted on the handle over the proximal bendable member; a rotation control member may be provided adjacent to the slide member for controlling the orientation of the distal movable member and tool.

In accordance with still another embodiment the medical instrument has a proximal control handle and a distal tool that are intercoupled by an elongated instrument shaft that is meant to pass internally of an anatomic body, proximal and distal movable members respectively intercouple the proximal control handle and the distal tool with the instrument shaft, cable actuation means is disposed between the movable members, for controlling the positioning of the distal tool, and sleeve means extends between the proximal and distal movable members and is slidable to a position over the distal movable member.

In accordance with still further aspects of the invention the sleeve means is preferably relatively rigid so as to fix the distal movable member in a predetermined position; the sleeve means preferably maintains the distal movable member in a straight position; a handle may be provided at the proximal end of the sleeve means to assist in moving the sleeve means and a locking mechanism may be provided having locked and unlocked positions, disposed about the proximal movable member and manually controlled so as to fix the position of the proximal movable member relative to the handle in the locked position thereof.

In accordance with still a further embodiment the medical instrument includes a proximal control handle; a distal work member; a proximal movable member controlled from the proximal control handle; a distal movable member controlled from the proximal movable member to provide controlled movement of the distal work member from the proximal control handle; an instrument shaft that intercouples the proximal and distal movable members and actuation means coupled between the movable members. The distal movable member comprises a ball and socket assembly that enables limited rotation of the distal work member relative to the distal end of the instrument shaft.

In accordance with another aspect of the invention a locking member is supported from the proximal control handle and has locked and unlocked states, said locking member in the unlocked state enabling control of the distal work member from the proximal control handle via the movable members, and said locking member, in its locked state, holding the movable members in a desired fixed position; the proximal movable member may comprise a proximal bendable member that includes a slotted structure; alternatively the proximal movable member may comprise a bendable bellows member and the actuation means may include cables and the ball may include plural recessed areas for accommodating the cables.

In a final embodiment the medical instrument has a proximal control handle and a distal tool that are intercoupled by an elongated instrument shaft that is meant to pass internally of an anatomic body, proximal and distal movable members respectively intercouple the proximal control handle and the distal tool with the instrument shaft, cable actuation means is disposed between the movable members and a locking means is manually operable by a user and includes a follower the position of which is responsive to the position of the movable members. The distal movable member comprises a ball and socket assembly that enables limited rotation of the distal work member relative to the distal end of the instrument shaft.

### BRIEF DESCRIPTION OF THE DRAWINGS

It should be understood that the drawings are provided for the purpose of illustration only and are not intended to define the limits of the disclosure. The foregoing and other objects and advantages of the embodiments described herein will become apparent with reference to the following detailed description when taken in conjunction with the accompanying drawings in which:

FIG. 1 is a perspective view of one embodiment of the surgical tool in which the instrument shaft and end effector can be rotated by means of a thumb slide;

FIG. 2 is a fragmentary cross-sectional view taken along line 2-2 of FIG. 1;

FIG. 3 is a cross-sectional view taken along line 3-3 of FIG. 2;

FIG. 4 is a schematic perspective view of the mechanism illustrated in FIGS. 2 and 3;

FIG. 5 is a schematic perspective view of an alternate embodiment of thumb slide mechanism;

FIG. 6 is a fragmentary perspective view of an alternate embodiment of the surgical tool in which the angle locking means is a split ball affixed to the handle of the instrument that is received in a socket affixed to the proximal end of the instrument shaft;

FIG. 7 is a fragmentary cross-sectional view at the proximal bendable member;

FIG. 8 is a cross-sectional view taken along line 8-8 of FIG. 7;

FIG. 9 is a schematic cross-sectional view of the mechanism seen in FIG. 8;

FIG. 10 is a fragmentary cross-sectional view taken along line 10-10 of FIG. 8;

FIG. 11 is a perspective view of an alternate embodiment of the surgical tool in which the distal bending member can be locked into a straight position by means of a slidable sleeve;

FIG. 12 is a fragmentary cross-sectional view through the instrument from the tip to the proximal bendable member;

FIG. 13 is a partial cross-sectional view of still a further embodiment of the instrument of the present invention illustrating a ball and socket arrangement at the distal end of the instrument;

FIG. 14 is a fragmentary perspective view of the ball and terminal end of the shaft of FIG. 13 by itself;

FIG. 15 is a cross-sectional side view of the distal bending member of FIG. 13 being used in an instrument with a proximal bending member of unitary construction; and

FIG. 16 is a cross-sectional side view of the distal bending member of FIG. 13 being used in an instrument with a ball-joint proximal bending member.

### DETAILED DESCRIPTION

The instrument of the present invention may be used to perform minimally invasive procedures. “Minimally invasive procedure,” refers herein to a surgical procedure in which a surgeon operates through a small cut or incision, the small incision being used to access the operative site. In one embodiment, the incision length ranges from 1 mm to 20 mm in diameter, preferably from 5 mm to 10 mm in diameter. This procedure contrasts those procedures requiring a large cut to access the operative site. Thus, the flexible instrument is preferably used for insertion through such small incisions and/or through a natural body lumen or cavity, so as to locate the instrument at an internal target site for a particular surgical or medical procedure. The introduction of the surgical instrument into the anatomy may also be by percutaneous or surgical access to a lumen, vessel or cavity, or by introduction through a natural orifice in the anatomy.

In addition to use in a laparoscopic procedure, the instrument of the present invention may be used in a variety of other medical, surgical or therapeutic procedures including, but not limited to, colonoscopic, upper GI, arthroscopic, sinus, thorasic, prostate, transvaginal, orthopedic and cardiac procedures. Depending upon the particular procedure, the instrument shaft may be rigid, semi-rigid or flexible.

Although reference is made herein to a “surgical instrument,” it is contemplated that the principles of this invention also apply to other medical instruments, not necessarily for surgery, and including, but not limited to, such other implements as catheters, as well as diagnostic and therapeutic instruments and implements.

There are a number of unique features embodied in the instrument disclosed herein. For example, there is provided a locking mechanism that may employ either a locking wedge, locking sleeve or locking lever arrangement for maintaining the proximal and distal bendable members in a particular bent condition, or in other words locked in that position. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob to, in turn, control the orientation of the end effector.

Another feature of the present invention relates to the provision of a distal ball and socket arrangement that enables three dimensional angled control of the instrument tool.

Still another feature of the present invention relates to a further form of control of the distal tool rotation via a proximal control member that operates on a motion basis that, in turn, through a mechanical interface, controls rotation of the tool about a distal tool axis.

Still another feature of the present invention is to provide a medical instrument in which there is provided a means, preferably a sleeve means, to readily convert the instrument tip into a substantially rigid tip. This is accomplished by sliding the sleeve over the instrument tip, particularly over the distal moveable member, to hold the tip in a particular position, preferably a straight position.

In the surgical instrument described herein both the tool and handle motion members or bendable members are preferably capable of bending in any direction. They are interconnected via cables (preferably four cables) in such a way that a bending action at the proximal member provides a related bending at the distal member. The proximal bending is controlled by a motion or deflection of the control handle by a user of the instrument. In other words the surgeon grasps the handle and once the instrument is in position any motion (deflection) at the handle immediately controls the proximal bendable member which, in turn, via cabling controls a corresponding bending or deflection at the distal bendable member. This action, in turn, controls the positioning of the distal tool.

The proximal member is preferably generally larger than the distal member so as to provide enhanced ergonomic control. In the illustrated embodiment the ratio of proximal to distal bendable member diameters may be on the order of three to one. In one version in accordance with the invention there may be provided a bending action in which the distal bendable member bends in the same direction as the proximal bendable member. In an alternate embodiment the bendable, turnable or flexible members may be arranged to bend in opposite directions by rotating the actuation cables through 180 degrees, or could be controlled to bend in virtually any other direction depending upon the relationship between the distal and proximal support points for the cables.

As has been noted the, amount of bending motion produced at the distal bending member is determined by the dimension of the proximal bendable member in comparison to that of the distal bendable member. In the embodiment described the proximal bendable member is generally larger than the distal bendable member, and as a result, the magnitude of the motion produced at the distal bendable member is greater than the magnitude of the motion at the proximal bendable member. The proximal bendable member can be bent in any direction (about 360 degrees) controlling the distal bendable member to bend in either the same or an opposite direction, but in the same plane at the same time. Also, the surgeon is able to bend and roll the instrument's tool about its longitudinal axis P to any orientation simply by rolling the axial rotation knob about rotation axis.

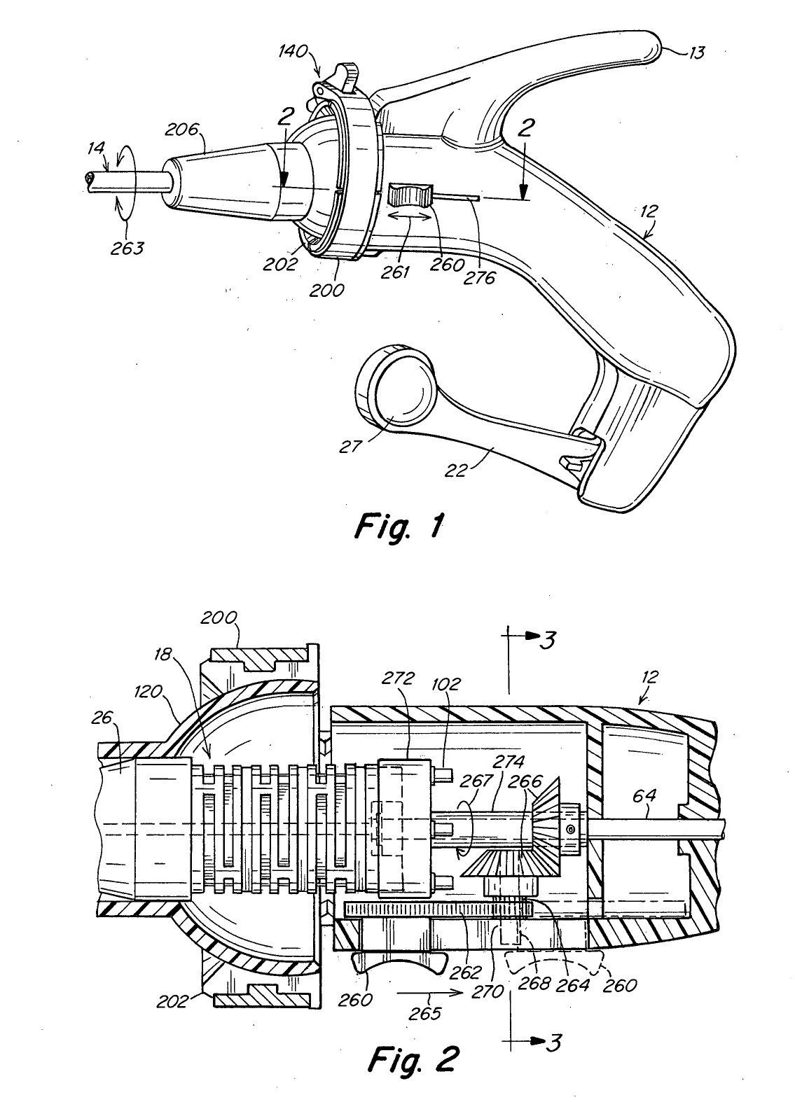

In this description reference is made to bendable members. These members may also be referred to as turnable members, bendable sections, bendable segments or flexible members. In the descriptions set out herein, terms such as “bendable section,” “bendable segment,” “bendable member,” or “turnable member” refer to an element of the instrument that is controllably bendable in comparison to an element that is pivoted at a joint. The term “movable member” is considered as generic to bendable sections and joints. The bendable elements of the present invention enable the fabrication of an instrument that can bend in any direction without any singularity and that is further characterized by a ready capability to bend in any direction, all preferably with a single unitary or uni-body structure. A definition of a “unitary” or “uni-body” structure is—a structure that is constructed only of a single integral member and not one that is formed of multiple assembled or mated components—. A typical unitary structure is illustrated at the proximal end of the instrument shown in FIG. 2.

A definition of these bendable members is—an instrument element, formed either as a controlling means or a controlled means, and that is capable of being constrained by tension or compression forces to deviate from a straight line to a curved configuration without any sharp breaks or angularity—. Bendable members may be in the form of unitary structures, such as shown herein in FIG. 2, may be constructed of engageable discs, or the like, may include bellows arrangements or may comprise a movable ring assembly. For other forms of bendable members refer to co-pending application Ser. Nos. 11/505,003 filed on Aug. 16, 2006 and 11/523,103 filed on Sep. 19, 2006, both of which are hereby incorporated by reference herein in their entirety. Also incorporated by reference in their entirety is Ser. No. 10/822,081 filed on Apr. 12, 2004; Ser. No. 11/185,911 filed on Jul. 20, 2005; Ser. No. 11/242,642 filed on Oct. 3, 2005 and Ser. No. 11/605,694 filed on Nov. 28, 2006.

The instrument disclosed herein may be used, for example, for laparoscopic surgery through the abdominal wall. For this purpose there is provided an insertion site at which there is disposed a cannula or trocar. The shaft of the instrument is adapted to pass through the cannula or trocar so as to dispose the distal end of the instrument at the operative site. The end effector may be considered as at such an operative site with the cannula or trocar at an incision point in the skin. The distal end of the instrument may be typically used with a sheath to keep bodily fluids from entering the distal bending member.

One embodiment of the surgical tool of the present invention is shown in FIG. 1-4. Actuation of the thumb slide 260 in the direction of the arrow 261 results in rotational movement of the shaft 14 and end effector in the direction of arrow 263. In related Ser. No. 11/528,134 filed on Sep. 27, 2006 and which is hereby incorporated by reference herein in its entirety, the tip rotation is illustrated as controlled by a rotation knob that, in turn, rotates the proximal bendable member and instrument shaft. As seen in FIG. 2 herein, the rotational knob is replaced by the slide switch 260 that is supported for sliding motion along the slot 276. The slide switch provides a more concentrated form of control and is easily manipulated with a single finger or the thumb. A collet 272 is attached to a hub 274 that is rotatable on the center wire conduit 64, as shown in FIG. 2. The collet 272 supports the proximal end of the proximal bendable member 1S. This embodiment also illustrates an angle locking means 140, as well as the cinch ring 200 (lock), split ball 202, actuation lever 22 and handle horn 13. Further details of the locking means and cinch arrangement are shown in Ser. No. 11/649,352 which is hereby incorporated by reference in its entirety.

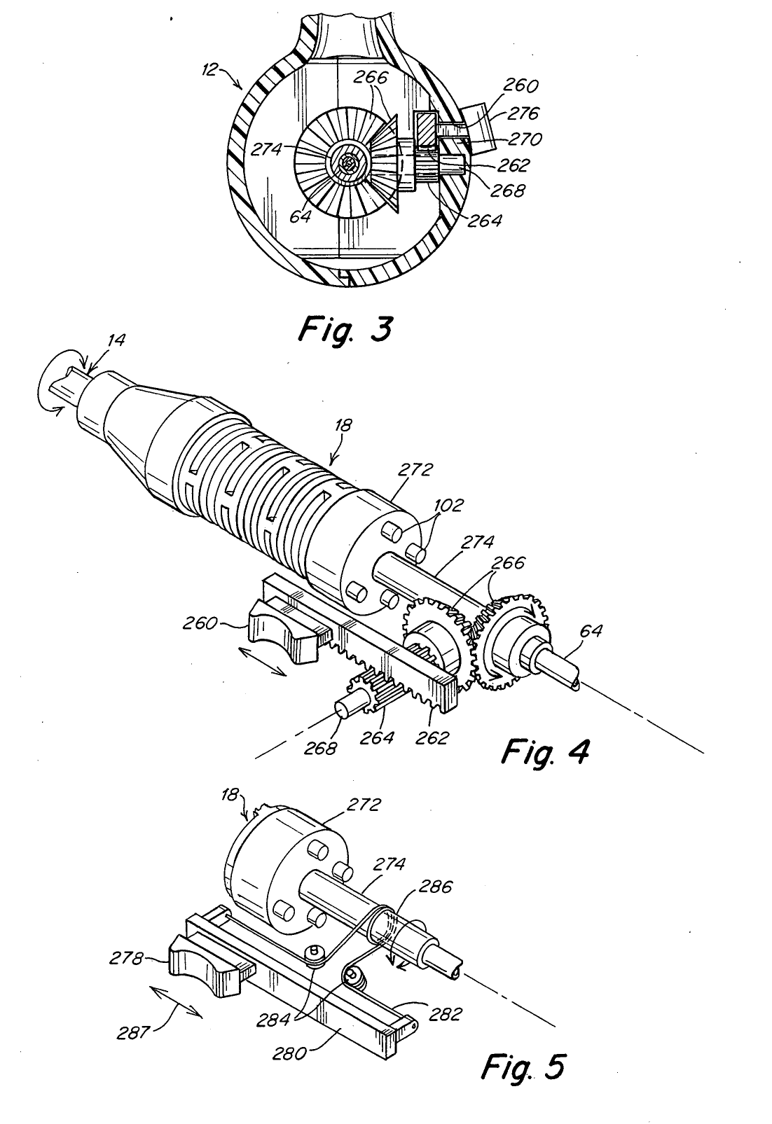

The thumb slide switch 260 is longitudinally slidable in slot 276 on the side of the handle 12. The thumb slide is affixed to a rack 262 that, in turn, rotates pinion gear 264 as it is pushed forward or rearward. The pinion gear 264 is affixed to or formed as part of a bevel gear 266 and the pinion gear 264 and one of the bevel gears are free to rotate on stub shaft 268. The stub shaft 268 is mounted in boss 270 (FIG. 3) that also acts as a slideway for the slide switch 260. When the slide switch 260 is pushed in the direction of arrow 265 in FIG. 2, the rack 262 urges the pinion gear 264 to rotate clockwise as seen from the bottom of the drawing. This rotates the hub 274 in the direction of arrow 267 by means of bevel gears 266. The thumb slide 260, as shown, is for a right handed person but may be placed on the opposite side of the handle for a left handed person or may be placed elsewhere on the handle.

An alternate drive means for performing the rotation function is shown in FIG. 5. The thumb slide switch 278 in this embodiment is attached to a spreader bar 280 that anchors cable 282. The cable 282 is attached at ends thereof to opposite ends of the bar 280. Forward and rearward movement of the thumb slide switch 278 drives cable 282 around pulleys 284 and capstan 286 to rotate the hub 274 and collet 272 around center wire conduit 64 as shown by the double headed arrows 287.

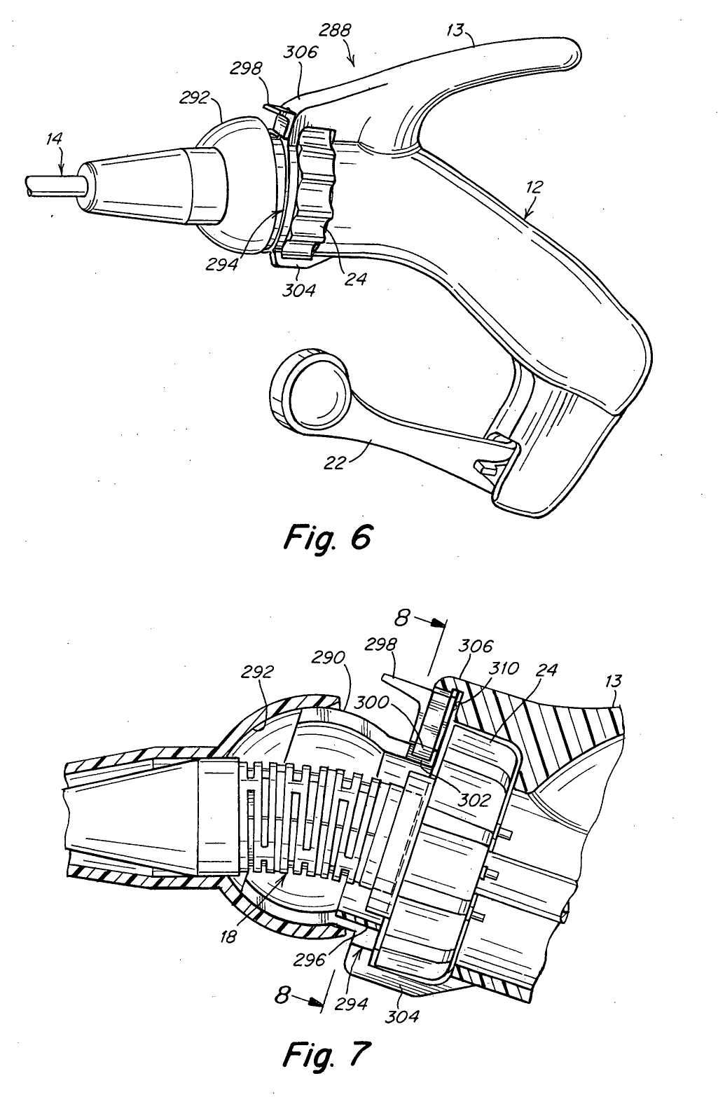

Reference is now made to a further embodiment of the invention shown in FIGS. 6-10. FIG. 6 shows this alternate embodiment 288 of the instrument in which the proximal bending member 18 includes a split ball 290 and split rim 294 affixed to the handle 12 and a socket 292 rotatably affixed to the instrument shaft 14. The embodiment of FIG. 6 also shows the handle horn 13 and actuation lever 22. The split ball 290 is formed as part of the split rim 294 which is, in turn, supported by two solid struts 304 and one split strut 306 from the handle. The struts have living hinges 308 (FIG. 9) which allow the expansion of the split ball 290 in the direction of arrows 303 when the wedge 300 is forced in the direction of arrow 301 (see FIG. 9). The split ball 290 and rim 294 are also allowed to flex outwardly by living hinge 296.

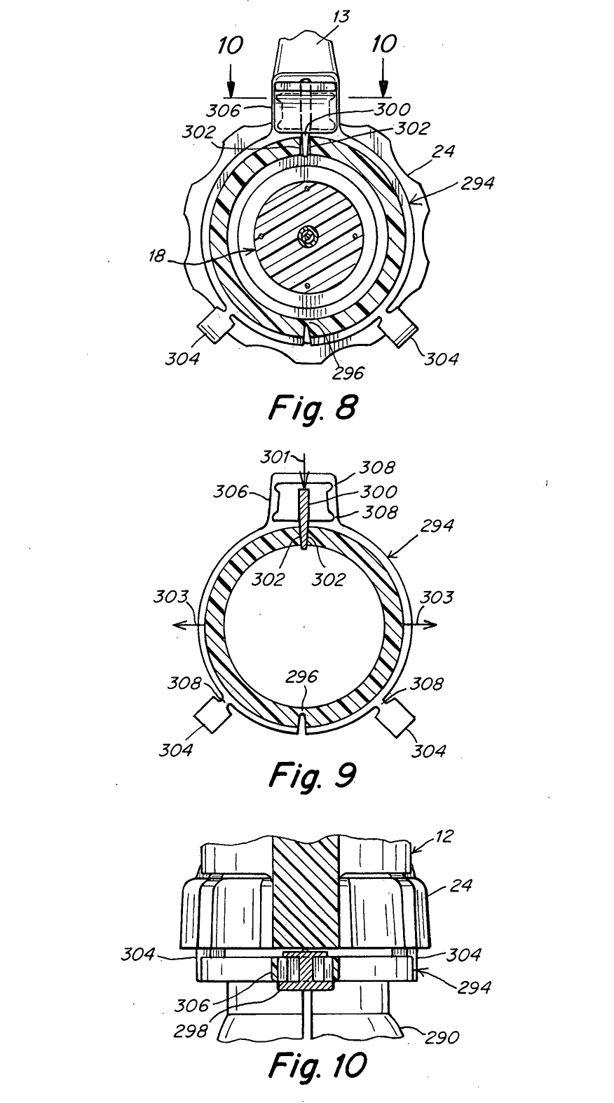

A thumb slide switch 298 drives the wedge 300 against cam surfaces 302 of the rim 294. This action, as seen in FIG. 8, spreads the split ball 290 against the socket 292 inner surface and thus locks the angle in place. The locking of the position of the proximal bendable member 18, in turn, locks the position of the distal end of the instrument. The thumb slide switch 298 may incorporate detents 310 so that the switch can be snapped into respective locked and unlocked positions. Even when the instrument is locked it is still possible to rotate the rotation knob 24 to rotate the proximal bendable member, instrument shaft and end effector about the distal tip axis P (as in FIG. 15).

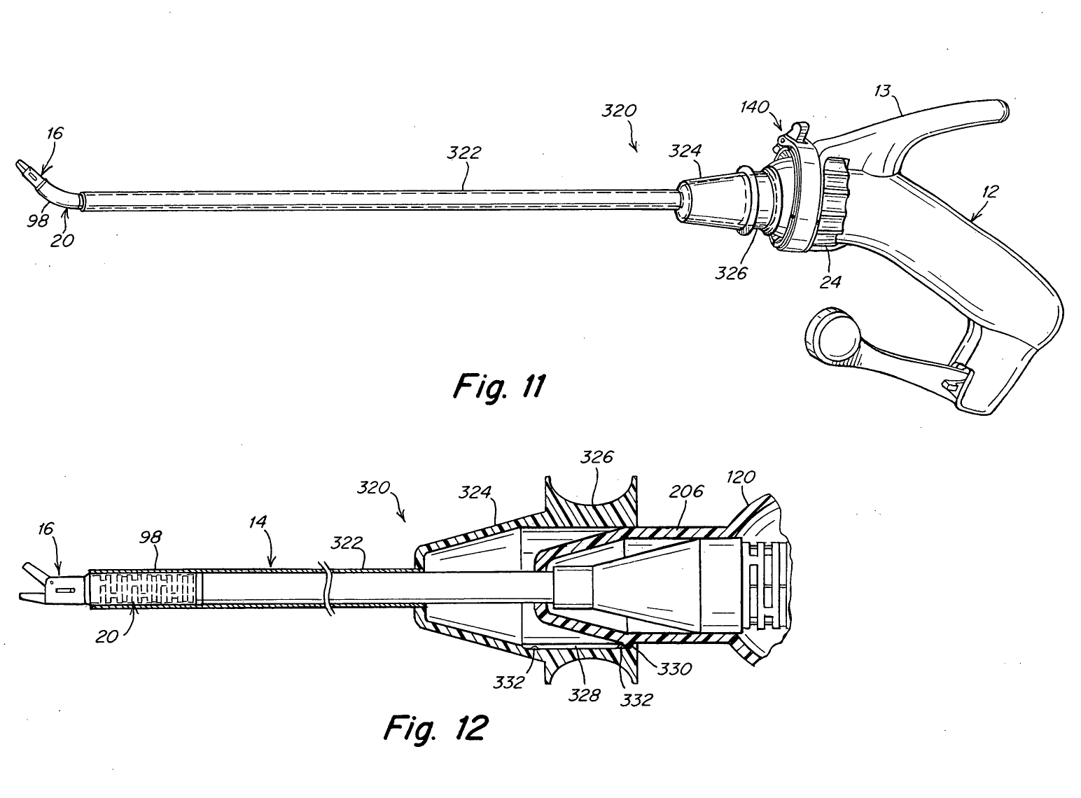

FIGS. 11 and 12 show still another embodiment 320 of the instrument in which the distal bending member 20 can be locked into a straight position as shown in FIG. 12 by pushing a rigid sleeve 322 forward over it and the sheath 98 if a sheath 98 is used. Although it is preferred to keep the tip straight, it is possible to have the structure of the sleeve somewhat flexible so that the distal member can be also held at a slight angular position, but still relatively rigid. For certain surgical applications it is desirable to be able to have a relatively rigid tip structure that will enable the surgeon to exert more force in a particular procedure. For normal operations the instrument shaft is rigid but the distal bendable member is bendable. By moving the sleeve 322 over the distal bendable member this makes the entire distal end of the instrument rigid and substantially straight.

The sleeve 322 is attached at its proximal end to handle 324 which has a gripping portion 326 and furthermore has a slot 328 on its inside surface that rides over one or more protusions 330 on the outside surface of the neck 206 of ball 120. The handle 324 may also have detents 332 to engage the handle in respective forward or rearward positions. FIG. 11 shows the sleeve 322 in a more rearward, proximal or retraced position, while FIG. 12 shows the sleeve 322 at an extended or distal position. In this more distal position it is noted that the sleeve has moved by moving the handle 324 distally with the gripping potion 326 shown moved along the neck 206. The sleeve 322 preferably rotates with any rotation that is imparted via the rotation knob 24.

In the embodiment of FIGS. 111 and 12 it is noted that the instrument also has the proximal angle locking means 140 such as shown in related co-pending application Ser. No. 11/649,352 which is hereby incorporated by reference herein in its entirety. This may be operated to lock the proximal bendable member and, in turn, the distal bendable member in a particular selected position.

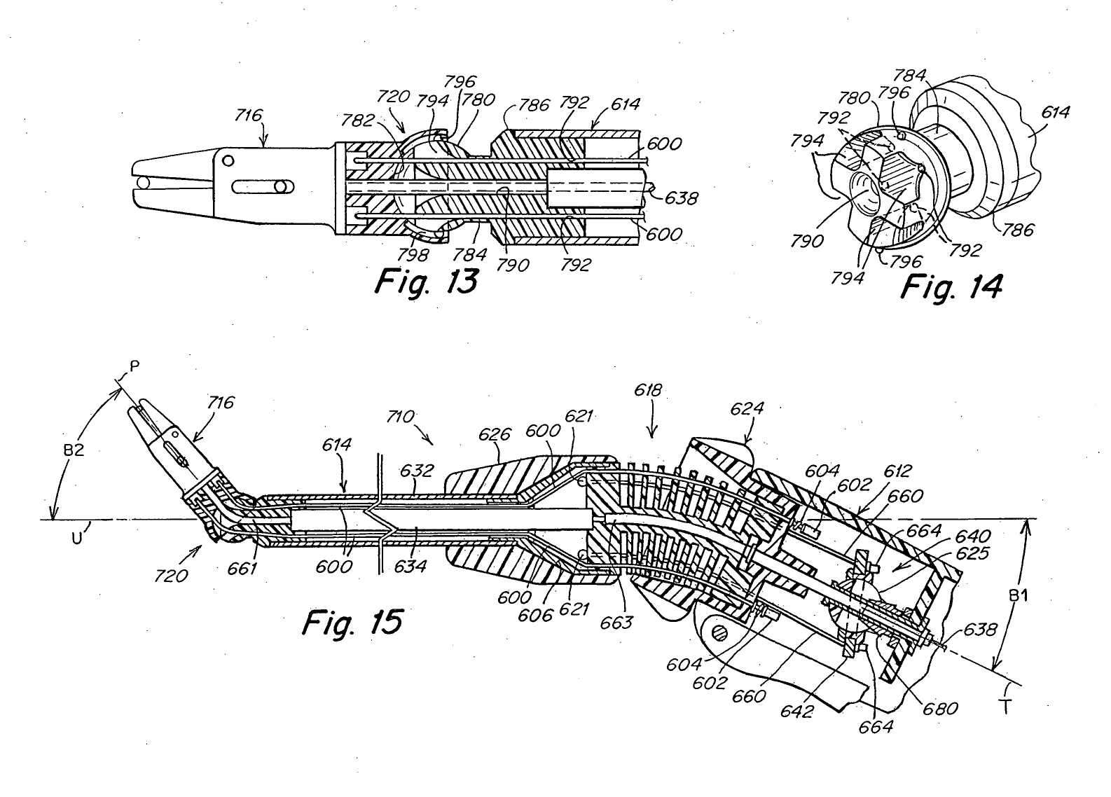

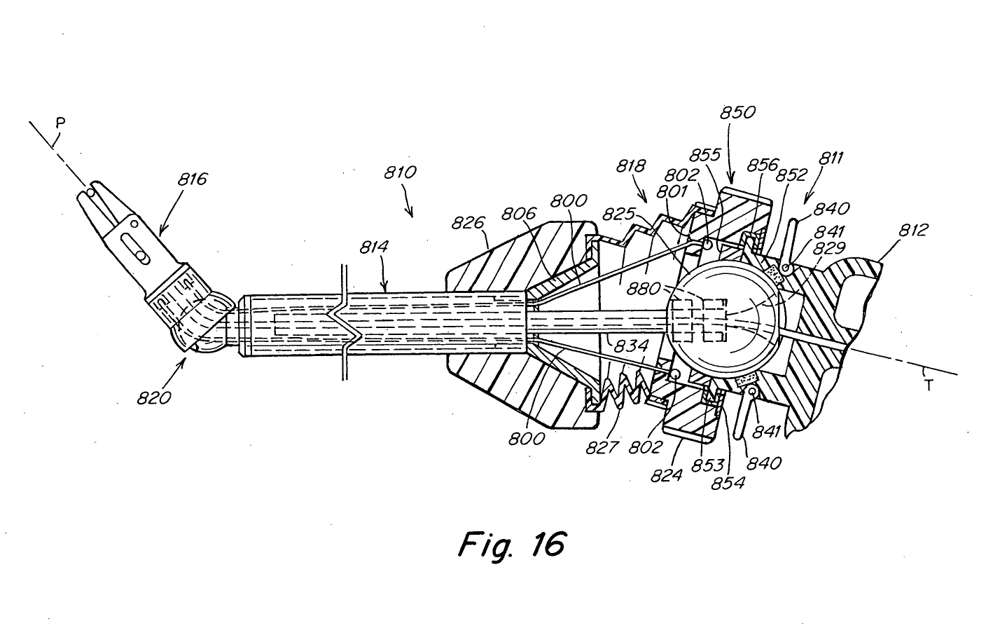

Reference is now made to other embodiments of the invention shown in FIGS. 13-16 in which the distal bendable member is formed by a ball and socket arrangement rather than a unitary slotted structure. FIG. 13 is a partial cross-sectional view illustrating a ball and socket arrangement at the distal end of the instrument. FIG. 14 is a fragmentary perspective view of the ball and terminal end of the shaft of FIG. 13 by itself. FIG. 15 is a cross-sectional side view of the distal bending member of FIG. 13 being used in an instrument with a proximal bending member of unitary construction. FIG. 16 is a cross-sectional side view of the distal bending member of FIG. 13 being used in an instrument with a ball-joint proximal bending member.

A rolling motion can be carried out with any one of the instruments disclosed herein. For example, in the embodiment of FIG. 15 this can occur by virtue of the rotation of the rotation knob 624 relative to the handle 612 about axis T. When the rotation knob 624 is rotated, in either direction, this causes a corresponding rotation of the instrument shaft 614. This same motion also causes a rotation of the distal bendable member and end effector 716 about an axis that corresponds to the instrument tip, depicted in FIG. 15 as about the longitudinal tip or tool axis P.

Any rotation of the rotation knob 624 while the instrument is locked (or unlocked) maintains the instrument tip at the same angular position, but rotates the orientation of the tip (tool). For a further explanation of the tip rotational feature refer to co-pending application Ser. No. 11/302,654, filed on Dec. 14, 2005, particularly FIGS. 25-28, which is hereby incorporated by reference in its entirety.

The handle 612, via proximal bendable member 618, may be tilted at an angle to the instrument shaft longitudinal center axis. This tilting, deflecting or bending may be considered as in the plane of the paper. By means of the cabling this action causes a corresponding bend at the distal bendable member 720 to a position wherein the tip is directed along an axis and at a corresponding angle to the instrument shaft longitudinal center axis. The bending at the proximal bendable member 618 is controlled by the surgeon from the handle 612 by manipulating the handle in essentially any direction including in and out of the plane of the paper in FIG. 15. This manipulation directly controls the bending at the proximal bendable member. Refer to FIG. 15 in which there is shown the axis U corresponding to the instrument shaft longitudinal axis. Refer also to the proximal bend angle B1 between axes T and U, and the corresponding distal bend angle B2 between axes U and P.

Thus, the control at the handle is used to bend the instrument at the proximal motion member to, in turn, control the positioning of the distal motion member and tool. The “position” of the tool is determined primarily by this bending or motion action and may be considered as the coordinate location at the distal end of the distal motion member. Actually, one may consider a coordinate axis at both the proximal and distal motion members as well as at the instrument tip. This positioning is in three dimensions. Of course, the instrument positioning is also controlled to a certain degree by the ability of the surgeon to pivot the instrument at the incision point. The “orientation” of the tool, on the other hand, relates to the rotational positioning of the tool, from the proximal rotation control member, about the illustrated distal tip or tool axis P.

In the drawings a set of jaws is depicted, however, other tools or devices may be readily adapted for use with the instrument of the present invention. These include, but are not limited to, cameras, detectors, optics, scope, fluid delivery devices, syringes, etc. The tool may include a variety of articulated tools such as: jaws, scissors, graspers, needle holders, micro dissectors, staple appliers, tackers, suction irrigation tools and clip appliers. In addition, the tool may include a non-articulated tool such as: a cutting blade, probe, irrigator, catheter or suction orifice.

As depicted in FIGS. 13 and 14, the end effector 716 is gimbaled on a ball 780 which is seated in socket 782 formed at the proximal end of the end effector. The ball 780 is supported on a neck 784 of a terminal end 786 of the instrument shaft 614. A central lumen 790 is provided through the terminal end 786 for accommodating the passage of tool actuation cable 638. End 786 of the instrument shaft is also provided with four lumens 792 for receiving respective cables 600 and corresponding dished-out areas 794 that allow for bending of the push pull cables when the end effector is bent at an angle B2. The ball 780 has two pins 796 which register with grooves 798 in the socket 782 to coordinate rotation of the end effector and the instrument shaft.

Reference is now made to FIG. 15 for an illustration of one embodiment of a medical instrument that incorporates the distal ball and socket arrangement shown in FIGS. 13 and 14. This instrument includes cabling for controlling the distal bendable member from the proximal bendable member and also includes additional cabling at the proximal bendable member for controlling a means for locking the bendable members in a fixed relationship. This illustrated embodiment uses a uni-body structure at the proximal bendable end of the instrument. For further details of the instrument of FIG. 15, particularly at the proximal end thereof refer to co-pending application Ser. No. 11/523,103 filed on Sep. 19, 2006 and which is hereby incorporated by reference in its entirety.

In the embodiment of FIG. 15 the surgical instrument 710 is comprised of a handle 612 at the proximal end of the instrument, an elongated instrument shaft 614 and a tool or end effector 716 that is disposed at the distal end of the surgical instrument. The surgical instrument shaft is usually rigid for laparoscopic procedures, typically constructed of a metal material. For intraluminal procedures the instrument shaft may be at least partially flexible or bendable. In this embodiment, the handle 612 may be comprised of two handle halves. A lever is manipulated by the surgeon for opening and closing the end effector 716 at the distal end of the instrument shaft 614. The end effector 718 is comprised of a movable jaw and a fixed jaw. The rotation knob 624 at the proximal end of the instrument shaft is used to rotate the entire instrument shaft and end effector. An adaptor cover 626 partially retains a portion of the proximal bendable member 618 as shown in FIG. 15.

As indicated previously, the end effector or tool 716 is actuated by means of a jaw actuation member including an elongated lever. The lever is supported from the handle housing. This operates the tool actuator cable 638 from a slider (not shown) in the handle housing. When the cable 638 is moved to the right, then the jaws are moved toward a closed position. In FIG. 15 the jaws are illustrated as closed grasping a needle.

The instrument shaft 614 includes an outer shaft tube 632 that may be constructed of a lightweight metal material or may be a plastic material. The proximal end of the tube 632 is received by the adaptor cover 626. The distal end of the tube 632 is secured to the distal bendable member 720. Within the outer shaft tube 632 there is provided a support tube 634 that is preferably constructed of a plastic material. Tube 634 extends between the distal bendable member 720 and the proximal bendable member 618. The jaw actuator cable 638 extends within this support tube 634. The support tube 634 may have disposed along its length a series of spacers (not shown). Each of the spacers is preferably evenly spaced along the instrument shaft and may be provided with slots for accommodating the tool actuator cables.

FIGS. 13 and 15 also illustrate the bending control cables 600 extending between the proximal bendable member 618 and the distal motion member 720. The jaw actuator cable 638 terminates at its distal end at the end effector. Within each of the members 618 and 720 there is provided a plastic tube. This includes a distal tube and a proximal tube 663. Both of these tubes may be constructed of a plastic such as polyethyletherkeytone (PEEK). The material of the tubes is sufficiently rigid to retain the cable and yet is flexible enough so that it can readily bend with the bending of the bendable members. The tubes are longitudinally stiff, but laterally flexible.

As indicated previously, the control between the proximal member 618 and the distal member 720 is carried out by means of the flex control cables 600. There are four such cables. At the distal end of these cables, as mentioned before, the cables connect to anchors at the jaw end of the instrument. The cables 600 are retained at there proximal ends by cable end lugs 602 terminating at the proximal end of the proximal member. Preferably springs 604 or other resilient members are retained between these end lugs 602 and a wall of the rotation knob 624. The springs 604 tension or take up the slack on the cables. Within the adaptor cover 626, the cables 600 extend through the transition member 606. The cables then extend to a larger diameter outer locus as they extend through the proximal bendable member. The stepped transition member 606 may be of metal and is secured to the end of the tube 632.

In the embodiment of the invention illustrated in FIGS. 13-15 the locking occurs by means of the use of a separate follower member illustrated as locking mechanism 640. This follower mechanism operates in conjunction with lock cables 660 to lock a particular position of the proximal bendable member, and by doing so also locking the position of the distal bendable member, as the proximal and distal members are interconnected by actuation cables 600.

The locking mechanism 640 includes, inter alia, an anchor ring 642 that provides the primary support for the locking cables 660, as well as the support of the locking mechanism from the rotation knob structure. In this regard, the anchor ring 642 includes diametrically disposed pins that are accommodated in elongated slots of opposed rearwardly extending fingers. The cables 660 are relatively rigid and generally of a larger diameter than the cables 600. All of the cables 600 are preferably of the same length.

When the instrument illustrated in this embodiment is in a straight in-line position then the locking mechanism, and particularly the anchor ring 642 extends substantially transverse to the center axis. When the handle 612 is bent, such as in the positions shown in FIG. 15 then it is noted that the follower locking mechanism 640 tilts relative to the longitudinal axis T. When it is desired to lock the mechanism in a particular bent condition then the wedge member 680 engages the split ball 625 and this locks the position of the anchor ring 642 and thus also locks the position of the locking or anchor cables 660. This, in turn, locks the position of the proximal bendable member 618 and via the cables 600 also locks the position of the distal member 720. The rigidity of the locking cables 660 maintains the proximal bendable member 618 in the locked position. The wedge member 680 may be actuated by means such as disclosed in the aforementioned Ser. No. 11/523,103.

Each of the cables 660 are disposed 90 degrees apart, as are the bent cables 600. The cables 660 may be disposed 45 degrees to the cables 600. The distal end of each cable 660 terminates at lug end 621. Rotation of the rotation knob 624 causes rotation of the entire proximal bendable member and the locking mechanism 640.

Reference is now made to FIG. 16 for a description of another instrument arrangement that employs the ball and socket distal member of FIGS. 13 and 14. The surgical instrument is comprised of a handle 812 at the proximal end of the instrument, an elongated instrument shaft 814 and a tool or end effector 816 disposed at the distal end of the surgical instrument. The embodiment of FIG. 16 also illustrates an adaptor cover 826 for partially retaining a portion of the proximal bendable member 818. At the distal end of the instrument shaft 814, there is provided the ball and socket member 820, such as illustrated in FIGS. 13 and 14. For further details of particularly the proximal end of the instrument refer to the aforementioned Ser. No. 11/523,103.

In the embodiment of FIG. 16, the ball 825 is attached to the proximal end of the instrument shaft and thus the handle 812 can bend or deflect at its rider 852. The axis of the handle shifts as the rider rotates on the ball 825. This action bends the proximal bendable member 818, such as to the position shown in FIG. 16. The positioning of the end effector is set or controlled by changing the angle of the handle relative to the instrument shaft. Once the tool is in the desired position then the locking mechanism 811 clamps the ball and socket (rider) together.

The proximal motion member 818 is constructed primarily of a bellows 827 that functions with the rotation knob 824 and locking mechanism 811 to control the distal end of the instrument. The bellows 827 is attached at opposite ends to the adaptor 826 at member 806 and at the rotation knob 824. The ends of the bellows may be secured by a compression fit with the respective adaptor 826 and rotation knob 824. As illustrated in FIG. 16 the bellows are constructed as an accordion pleat and have a relatively rigid construction so that they are relatively stiff in the rotational direction, and yet are readily flexible (foldable) in the longitudinal direction. Any rotation imparted to the rotation knob 824 is coupled via the bellows 827 to the adaptor 826 and instrument shaft 814, and from there to the distal end of the instrument to rotate the end effector. Thus, in this embodiment the handle is tilted on the ball to control the proximal bendable member which, in turn, controls the position of the distal bendable member. While in any bent position the rotation knob is used to rotate the tip of the instrument about the tip axis P.

In the embodiment of FIG. 16 the position or location of the end effector is set by means of a ring assembly 850 that may be considered as including, inter alia, the aforementioned rotation knob 824, as well as the levers 840, annular cable retainer 801 and locking mechanism 811. This ring assembly 850 also includes the rider 852 with its flange 853, retaining rings 854 and 855, bearing 856 and fasteners. The ring assembly 850 is manipulated via the rotation knob 824 to control the cabling to the end effector and in a pitch and yaw manner. This action pushes and pulls the cabling to set the position of the end effector. At the same time the rotation knob 824 may be rotated to rotate the tip of the instrument about its tip axis. See axis P in FIG. 15. In FIG. 16 the levers 840 are shown in their unlocked position to thus unclamp the ring assembly 850 relative to the ball 825. Any rotation of the rotation knob 824 while the instrument is locked (or unlocked) maintains the instrument tip at the same angular position, but rotates the instrument orientation of the tip of the instrument at the end effector.

Refer now to FIG. 16 for further details of the ring assembly 850. The annular cable retainer 801 and rotation knob 824 form a unit that may be constructed of one or separate parts. This unit is rotational by means of the bearing or bushing 856 relative to the flange 853 of the rider 852. The annular rider 852 may be secured with the retaining ring 855 by means of one or more securing screws. Any rotation of the rotation knob 824 causes a rotation of the cable retainer 801 and the associated cables 800. FIG. 16 also shows the cables terminating at their proximal ends at the end lug 802. A spring or resilient member may be associated with each securing lug 802. The rider 852 and retaining ring 855 capture the shaft ball 825 and have their inner surfaces conform to the shape of the spherical ball 825. The rider 852 is supported so as to be free to pitch and yaw on the ball 825, while the rotation knob 824 is free to rotate relative to the rider 852. A second retainer 854 is fastened to the knob 824 about the flange 853. The user of the instrument can manipulate the rotation knob and rider separately with separate fingers. FIG. 16 shows the ring assembly 850 tilted to provide a like tilt of the end effector.

FIG. 16 also shows, in dotted outline, the bearings 880 that enable relative rotation between the inner shaft 834 and the ball 825 when the instrument shaft is rotated from the rotation knob 824. The inner shaft 834 may have a proximal end collar for positioning and supporting the inner shaft relative to the bearings. A thrust washer may also be provided between the bearing 880 and collar. The handle may be provided in two halves joined by locating pins in the ball 825; FIG. 16 also shows an expanded or flared channel at 829 in the ball 825. This configuration assists in the free rotation of the handle to enable a bending of the actuator cable tube, such as in a position illustrated in FIG. 16.

The embodiment described in FIG. 16 also includes a lock feature that enables the relative position between the proximal and distal motion members to be fixed in a predetermined position, such as the position illustrated in FIG. 16 where the rotation knob and lock mechanism have been bent, pivoted or rotated causing a corresponding bending of the tool. Once the surgeon has the instrument in the desired bent position then the locking mechanism is used to conveniently hold the instrument in that position. However, even though locked, the tool orientation can be changed via the rotation knob, or the like. The specific locking member is shown in FIG. 16 as provided by the opposed lock levers 840 that are pivotally supported at pins 841. FIG. 16 shows the both lock levers 840 in their released position in which the bendable members are permitted to bend in the normal operation of the instrument without being locked. The levers 840 are pivoted toward the rider 852 to urge the lever pad into engagement with the ball 825.

In this embodiment although a pair of lock levers is illustrated it is understood that only a single lock lever may be used. When a pair of lock levers is used they are normally both held in the same position, either locked or unlocked. This locking feature is an important adjunct to the other controls of the instrument enabling the surgeon to lock the instrument once in the desired position. This makes it easier for the surgeon to thereafter perform surgical procedures without having to, at the same time, hold the instrument in a particular bent configuration. However, even when locked, the end effector can still be rotated to control tool orientation.

While this invention has been particularly shown and described with references to preferred embodiments thereof, it will be understood by those skilled in the art that various changes in form and details may be made therein without departing from the scope of the invention encompassed by the appended claims. For example, the embodiments described herein have primarily used four control cables for providing all direction motion of the motion members. In alternate embodiments fewer or greater numbers of cables may be provided. In a most simplified version only two cables are used to provide single DOF action at the bendable motion member. Also, any of the disclosed embodiments can use a handle that is either essentially in line with the instrument shaft or of a pistol grip type.

What is claimed is:

1. An instrument having a proximal control handle and a distal tool that are intercoupled by an elongated instrument shaft, proximal and distal movable members that respectively intercouple said proximal control handle and said distal tool with said instrument shaft, cabling that extends between said movable members so that a motion at said proximal movable member controls said distal movable member, and a control member at said control handle and manipulable by a user to control, via said proximal and distal movable members, the rotation of said distal tool about its distal tool axis, said control member comprising a slide member mounted on said handle.

2. The instrument of claim 1 wherein said slide member moves longitudinally to, in turn, control the rotation about a distal tool axis that is a longitudinal axis common to both said distal movable member and said tool.

3. The instrument of claim 1 wherein said proximal movable member comprises a proximal bendable member.

4. The instrument of claim 1 including an angle locking means on the handle for locking the angle between the proximal and distal movable members.

5. The instrument of claim 1 wherein said movable members are bendable members and said control member further comprises gear means for translating linear sliding motion into rotation of said proximal bendable member.

6. The instrument of claim 1 wherein said movable members are bendable members and said control member further comprises a pulley and capstan means for translating linear sliding motion into rotation of said proximal bendable member.

7. In a medical instrument having a proximal control handle and a distal tool that are intercoupled by an elongated instrument shaft that is meant to pass internally of an anatomic body, proximal and distal movable members that respectively intercouple said proximal control handle and said distal tool with said instrument shaft, cable actuation means disposed between said movable members, for controlling the positioning of said distal tool, and a locking mechanism having locked and unlocked positions, disposed about said proximal movable member and manually controlled so as to fix the position of said proximal movable member relative to said handle in said locked position thereof, said locking mechanism comprising a socket and a split ball within said socket

8. The medical instrument of claim 7 wherein said locking mechanism further comprises a wedge member that engages with said split ball.

9. The medical instrument of claim 8 including a slide member for actuating said wedge member which, in turn, expands said split ball to contact said socket.

10. The medical instrument of claim 9 wherein said proximal movable member is a bendable member and said slide member is mounted on said handle over said proximal bendable member.

11. The medical instrument of claim 10 including a rotation control member adjacent to said slide member for controlling the orientation of said distal movable member and tool.

12. In a medical instrument having a proximal control handle and a distal tool that are intercoupled by an elongated instrument shaft that is meant to pass internally of an anatomic body, proximal and distal movable members that respectively intercouple said proximal control handle and said distal tool with said instrument shaft, cable actuation means disposed between said movable members, for controlling the positioning of said distal tool, and sleeve means extending between said proximal and distal movable members and slidable to a position over said distal movable member.

13. The medical instrument of claim 12 wherein said sleeve means is relatively rigid so as to fix the distal movable member in a predetermined position.

14. The medical instrument of claim 13 wherein said sleeve means maintains the distal movable member in a straight position.

15. The medical instrument of claim 13 including a handle at the proximal end of said sleeve means to assist in moving said sleeve means.

16. The medical instrument of claim 12 including a locking mechanism having locked and unlocked positions, disposed about said proximal movable member and manually controlled so as to fix the position of said proximal movable member relative to said handle in said locked position thereof.

17. A medical instrument comprising:
a proximal control handle;
a distal work member;
a proximal movable member controlled from said proximal control handle;
a distal movable member controlled from said proximal movable member to provide controlled movement of said distal work member from said proximal control handle;
an instrument shaft that intercouples said proximal and distal movable members;
and actuation means coupled between said movable members;
said distal movable member comprising a ball and socket assembly that enables limited rotation of the distal work member relative to the distal end of the instrument shaft.

18. The medical instrument of claim 17 further including a locking member supported from said proximal control handle and having locked and unlocked states; said locking member in said unlocked state enabling control of said distal work member from said proximal control handle via said movable members; and said locking member, in said locked state, holding said movable members in a desired fixed position.

19. The medical instrument of claim 17 wherein said proximal movable member comprises a proximal bendable member that includes a slotted structure.

20. The medical instrument of claim 17 wherein said proximal movable member comprises a bendable bellows member.

21. The medical instrument of claim 17 wherein said actuation means includes cables and said ball includes plural recessed areas for accommodating said cables.

22. In a medical instrument having a proximal control handle and a distal tool that are intercoupled by an elongated instrument shaft that is meant to pass internally of an anatomic body, proximal and distal movable members that respectively intercouple said proximal control handle and said distal tool with said instrument shaft, cable actuation means disposed between said movable members and a locking means that is manually operable by a user and that includes a follower the position of which is responsive to the position of said movable members, said distal movable member comprising a ball and socket assembly that enables limited rotation of the distal work member relative to the distal end of the instrument shaft.
